(19) United States
(12) Patent Application Publication
(10) Pub. No.: US 2015/0250966 A1
(43) Pub. Date: Sep. 10, 2015

(54) INTRODUCING DEVICE AND SLEEVE FOR LINING THE INNER SURFACE OF AN AIRWAY TUBE, SYSTEM AND METHOD FOR USING SAME

(71) Applicant: NOVO PATH LTD, Nazareth-Illit (IL)

(72) Inventor: Roni Shabat, Kfar Yehezkel (IL)

(73) Assignee: NOVO PATH LTD, Nazareth-Illit (IL)

(21) Appl. No.: 14/433,679

(22) PCT Filed: Oct. 10, 2013
(86) PCT No.: PCT/IL2013/050818

§ 371 Date: Apr. 6, 2015

Related U.S. Application Data
(60) Provisional application No. 61/711,752, filed on Oct. 10, 2012.

(51) Int. Cl. A61M 16/04

(52) U.S. Cl. CPC A61M 16/0488; A61M 16/0465; A61M 16/0486; A61M 16/0461; A61M 2205/02

(57) ABSTRACT

The present invention relates to ventilation tubes a device, system and method for lining ventilation tube with a removable inner sleeve.

### FIELD OF THE INVENTION

The present invention relates to ventilation tubes for ventilating and/or administrating drugs into the lungs, and in particular, to such a device, system and method for lining ventilation tube with a removable inner sleeve.

### BACKGROUND OF THE INVENTION

Endotracheal and tracheostomy tubes are two forms of airway tubes used to provide an open airway in patients who do not have an adequate airway flow and therefore require artificial ventilation, do due to various medical conditions. An endotracheal tube is inserted through the mouth and larynx and into the trachea. Tracheostomy tubes are inserted through an incision just above the sternal notch. Once in place, the now intubated patient may be ventilated mechanically, automatically or otherwise ventilated through the tube with various ventilation devices. An endotracheal tube (ETT) is typically a flexible tube inserted through the mouth or nose and utilized to gain access to the lower respiratory system and lungs. The ETT may be associated within the trachea for extended period of time in order to maintain an unobstructed passageway to allow for the delivery of flowing fluids, particularly gases, into the lungs. The flowing fluids introduced via an ETT include oxygen, anesthetics and various medications to be delivered to the lungs.

The ETT is also used to allow suctioning of mucus buildup within the tube, for example during ventilation. There are a number of risks in the prolonged use of an ETT as the mucus buildup, commonly referred to as bio-burden, builds along the internal wall of the ETT and could pose a threat to the patient. Such bio-burden could lead to the development of various ailments, for example, hypoxemia, tube blockage, and perhaps most importantly Ventilator-Associated Pneumonia (VAP).

VAP is potentially life threatening and is believed to be caused by bacterial build up in the moist environment on the ventilation tubing apparatus that causes infection to the patient's lungs. VAP is considered to be one of the more threatening hospital acquired disease that could lead to death in critically ill patients.

Accordingly precautions taken by medical teams to avoid VAP most of which consist of replacing the ETT tube to avoid the bacterial build up within the tube. However such placement and replacement of an airway tube such as an ETT is not a practical solution.

A breathing tube having an inner cannula that can be removed off the intubation tube and replaced with a new one is disclosed in US patent application the publication US20100108075 to Singvogel. However drawback of such system is that particles may fall off the cannula and get into the airways of the patient. The cannula disclosed by Singvogel significantly reduces the open lumen of the airway tube thereby requiring to ventilate a patient with increased ventilation pressure.

Another breathing aid which is a multi-layered breathing tube is disclosed in PCT Publication No. WO2011001437 to Tubular Medical Ltd. The sidewall of this breathing tube is lined with one or more lining sleeves where removing the innermost lining sleeve reveals the next sleeve that previously enclosed the sleeve that has been just removed off is exposed to the airways of the patient. A pulling wire that is tied to the distal end of each sleeve provide for sealing off its distal aperture prior to its being removed away. However such sealing renders the tube susceptible to the creation of a vacuum and resistance to removal that could lead to tearing of the lining sleeve and increased exposure to bio-burden in the lower airway tract.

### SUMMARY OF THE INVENTION

The present invention overcomes the deficiencies of the background by providing a device, system and method for inserting a removable single use inner sheath provided to line the internal surface of airway tubes so as to eliminate the need for airway tube replacement, enable immediate removal of a mucus plug or the like foreign body obstructing the airway tube and to reduce the risk of acquiring Ventilator Associated Pneumonia (VAP). The risk of VAP may be reduced by safely removing the removable single use inner sheath once it is soiled and thereafter replacing it with a new lining to sheath.

The lining sheath according to the present invention maintains an open lumen throughout its use so as to maximize the functional diameter of the airway tube so as to ensure that proper ventilation and ventilation pressure is utilized.

Embodiments of the present invention provide a multilayered breathing tube for example including but not limited to endotracheal tubes or tracheostomy tubes, including at least one single use lining sheath associated along the inner surface of the breathing tube.

Embodiments of the present invention provide for a single use airway tube lining sheath that may be securely associated and dissociated with the internal surface of an airway tube for example including not limited to an endotracheal tube or tracheostomy tube.

Embodiment of the present invention provide an introducing device provided for introducing and associating a single use lining sheath with the internal surface of an airway tube for example including but not limited to an Endotracheal tube or tracheostomy tube.

Embodiment of the present invention provide a method for removing a single use lining sheath disposed along the internal surface of an airway tube wherein the method of removal maintains the airway tube patency and reduces the risk of VAP by inverting the lining sheath.

Embodiments of the present invention provide a lining sheath having a tubular body including a wall defining an inner surface and an outer surface, the wall comprising an integrated removal thread.

Optionally the lining sheath's external surface comprises coupling enhancers provided to facilitate coupling between the sheath's external surface and the internal surface of the airway tube.

Embodiments of the present invention provide a device for introducing an airway tube sleeve for lining an airway tube, the device comprising: a handle for manipulating the device into an airway tube, an to introducing shaft extending distally from the handle and provided for associating with the sleeve; a sleeve retaining and releasing module for disassociating the sleeve from the shaft; and a sleeve coupling module disposed about the distal end of the shaft provided for facilitating coupling the sleeve onto the inner wall of the airway tube.

Optionally the shaft may be provided with recess along its length. Optionally the introducing shaft may be hollow.

Optionally the retaining and releasing module and coupling module may be provided as a single unit.

Optionally the retaining and releasing module may be disposed along the length of the introducing shaft. Optionally the retaining properties and/or functions may be provided along the length of the introducing shaft. Optionally the releasing properties and/or functions may be provided at the distal end of the introducing shaft.

Optionally the sleeve coupling module comprises a coupling member that preferably provides for urging the external surface of the airway tube lining sleeve onto the inner wall of the airway tube therein non-chemically coupling the two surfaces.

Optionally the surfaces of the airway tube and airway tube lining sleeve may be coupled by way of electrostatic forces.

Optionally the surfaces of the airway tube and airway tube lining sleeve may be coupled by way of van der Waals forces.

Optionally the coupling member may be provided in the form of a ring that may assume a small diameter when introduced and an expanded diameter when coupling the airway tube lining sleeve with the airway tube's inner surface.

Optionally the ring member may be provided from at least two semi-circular members.

Optionally the coupling member may be provided in the form of a claw having at least two manipulating fingers.

Optionally the coupling member may assume a small profile when to introducing the airway tube lining sleeve and an expanded profile when coupling the airway tube lining sleeve.

Optionally the coupling member may comprise a plurality of radially extending projection, for facilitating coupling of the airway tube lining sleeve the airway tube inner surface.

Optionally a proximal portion of the sleeve retaining and releasing module provides for unfolding the airway tube lining sleeve. Optionally the proximal portion provides for releasing the airway tube lining sleeve from the introducing shaft.

Optionally a distal portion of the coupling module may provide for urging the external surface of the airway tube lining sleeve onto the inner wall of the airway tube.

Optionally the introducing shaft comprises a recess along its length provided for receiving and housing the airway tube lining sleeve in a minimal profile configuration.

Optionally the sleeve retaining and releasing module may comprise a recess for retaining at least a portion of the distal portion of the airway tube lining sleeve.

An optional embodiment of the present invention provides an airway tube lining sleeve, the sleeve forming a substantially tubular body having an open lumen defined between a distal end and a proximal end, the proximal end affixed with an adaptor for coupling both with the machine end of an airway tube and with a ventilation device; the tubular body defining a wall, the wall having at least one intrinsic removal thread spanning the length of the tubular body and wherein the removal thread forms a noose spanning the perimeter of the sleeve's distal end; the noose provided for reducing the circumference of the distal end and allowing for the inversion of the lining sleeve during removal with the removal thread.

Optionally the airway tube lining sleeve may comprise a wall having a thickness of about 0.2 mm.

Optionally the wall may be configured to have thickness selected to from the group consisting of 0.25 mm, 0.50 mm, 0.075 mm, 0.1 mm, 0.125, 0.15 mm, 0.0175 mm, 0.2 mm, 0.225 mm, 0.25 mm, 0.275 mm, 0.3 mm, 0.325 mm, 0.35 mm, 0.375 mm, 0.4 mm, 0.425 mm, 0.45 mm, 0.475 mm, 0.5 mm, 0.525 mm, 0.55 mm, 0.575 mm, 0.6 mm, 0.65 mm, 0.7 mm, 0.75 mm, 0.8 mm, 0.85 mm, 0.9 mm, 0.95 mm, 1.0 mm, or the like.

Optionally the sleeve body may be provided from optional biocompatible materials for example including but not limited to any one or more materials selected from latex, silicone, medical grade polymers, biocompatible elastomers, medical grade silicone, Polyvinyl Chloride (PVC), PVC elastomers, fluorinated polymers (PTFE), polyolefin, polyurethane, Teflon, nylon, Nitrile butadiene rubber (NBR), soft PVC, polypropylene, any mixture thereof, any hybrid thereof, any composition thereof, any combination thereof.

Optionally the sleeve wall may further comprise supportive wire disposed along the length of the tube body. Optionally the supportive wire may be provided in the form of a helical wire looped along the length of the tube body.

The wall may be define between an inner surface and an outer surface, wherein the outer surface may be provided for coupling with the internal surface of an airway tube; and wherein the outer surface comprises coupling enhancers to enhance coupling and holding force between the lining sleeve and the airway tube.

Optionally the coupling enhancers may be provided in the form of protrusions extending from the external sleeve surface. Optionally the protrusions may assume a surface configuration for example including but not limited to any one or more of concave external surface, a convex external surface, extensions, projections, or the like preferably provided to increase the surface area of the external surface.

Optionally the lining sleeve adaptor may be provided from a plurality of subunits. Optionally the adaptor may be provided from at least two subunits. Optionally the at least two subunits comprise a first subunit coupled with the proximal end of the lining sleeve body and a second subunit coupled with the removal thread.

Optionally the adaptor may comprise an opening or recess through which the removal thread may be pulled.

Optionally the first subunit may comprise an opening through which the removal thread may be pulled.

Optionally the noose may be adapted to maintain an open lumen along the sleeve's length.

Optionally the noose may be adapted reduce the sleeve's diameter by up to about 75%.

Optionally the noose may be adapted reduce the sleeve's diameter by a factor selected from the group for example including but not limited to up to about 75%, 50%, 25%, or 15%.

An optional embodiment of the present invention provides a system for introducing an airway tube lining sleeve into an airway tube wherein the airway tube may be associated with a patient, the system comprising a sleeve introducing device,—and an airway tube lining sleeve.

An optional embodiment of the present invention provides a method for introducing an airway tube lining sleeve into an airway tube associated with a patient the method comprising: associating an airway tube lining sleeve according to the present invention with an introducing device according to the present invention; Introducing the shaft into the proximal end of the airway tube through to the distal end of the airway tube; Releasing the airway tube lining sleeve from the introducing device utilizing the releasing module; Non-chemically coupling the sleeve's external surface with the airway tube internal surface utilizing the sleeve coupling module to urge the external surface onto the airway tube internal surface from the distal end to the proximal end, with the extracting module and coupling module, as the introducing device may be extracted proximally out of the airway tube.

An optional embodiment of the present invention provides a method for removing an airway tube lining sleeve according to embodiments of the present invention—from an airway tube associated with a patient, the method comprising: Removing ventilation device from the adaptor; pulling the removal thread proximally to activate the noose to reduce the diameter of the lining sleeve to a controllable level while maintaining an open lumen along the length of the airway tube; continue pulling the removal thread proximally to invert the lining sleeve; stop pulling the removal thread when the inverted distal end reaches the adaptor; remove the inverted lining sleeve with the adaptor; and replace and re-associate airway tube with the ventilation device.

Optionally—the airway tube previously described may for example be selected from the group consisting of: Endotracheal tube (ETT), endobronchial tube, tracheostomy tubes, laryngeal tubes, nasopharyngeal tubes, single lumen tubes, double lumen tubes, multi-lumen tubes or the like.

Within the context of this application the term “airway tubes” is to refer to any medical tube utilized within the airway tract to maintain patency of the upper airways. The terms “airway tube” and may interchangeably be used with and may refer to any of the following terms Endotracheal tube (ETT), endobronchial tube, tracheostomy tubes, laryngeal tubes, nasopharyngeal tubes, single lumen tubes, double lumen tubes, multi-lumen tubes, or the like.

Unless otherwise defined, all technical and scientific terms used herein have the same meaning as commonly understood by one of ordinary skill in the art to which this invention belongs. The materials, methods, and examples provided herein are illustrative only and not intended to be limiting. Implementation of the method and system of the present invention involves performing or completing certain selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A is a schematic block diagram of an exemplary airway tube lining sleeve introducing device according to an optional embodiment of the present invention;

FIG. 1B is a schematic block diagram of an exemplary airway tube lining sleeve according to an optional embodiment of the present invention;

FIG. 2A-C are schematic illustrations of optional airway tube lining sleeves according to an optional embodiment of the present invention;

FIG. 3A-C are schematic illustrations of an exemplary airway tube lining sleeve introducing device according to an optional embodiment of the present invention, FIG. 3A shows a perspective view, FIG. 3B-C show a close up view of the distal end;

FIG. 4 is a schematic illustration of an exemplary airway tube lining sleeve introducing device according to an optional embodiment of the present invention;

FIG. 5A-C are schematic illustrations of an optional sleeve introduce and release module portion of an airway tube lining sleeve introducing device according to an optional embodiment of the present invention;

FIG. 6A-D show various views of schematic illustrations of an to optional sleeve introduce and release module portion of an airway tube lining sleeve introducing device according to an optional embodiment of the present invention;

FIG. 7A-D are schematic illustrations of an optional sleeve introduce and release module portion of an airway tube lining sleeve introducing device according to an optional embodiment of the present invention;

FIG. 8A-F are schematic illustrations of the various stages of removing an airway tube lining sleeve while maintaining patency of airway tube, according to an optional embodiment of the present invention;

FIG. 9 is a flowchart of an optional method for removing an airway tube lining sleeve according to an optional embodiment of the present invention; and

FIG. 10 is a flowchart of an optional method for introducing an airway tube lining sleeve according to an optional embodiment of the present invention.

### DESCRIPTION OF THE PREFERRED EMBODIMENTS

The principles and operation of the present invention may be better understood with reference to the drawings and the accompanying description.

The following figure reference labels are used throughout the description to refer to similarly functioning components used throughout the specification hereinbelow.
10 inner sleeve introducing system;
50 airway tube;
50d distal end of airway tube;
50p proximal end of airway tube;
100 sleeve introducing device;
100a direction of extraction of introducing device;
102 handle;
104 shaft;
104r shaft recess;
106 Sleeve retaining and releasing module;
106c C shaped shaft;
106a,b shaft flanking arms;
106d shaft pivot point
106e controller;
106r retaining recess;
108 sleeve coupling module;
108a coupling member low profile;
108e coupling member expanded profile;
108c coupling member
110 airway tube lining sleeve;
110p proximal end;
110d distal end;
112 sleeve-airway tube adaptor;
112a,b adaptor sub-units;
114 sleeve body;
114e sleeve external surface;
114i sleeve internal surface;
114w sleeve wall;
116 sleeve removal thread;
116n noose portion;
116p pull portion;
118 sleeve support wire;
120 sleeve coupling enhancer;

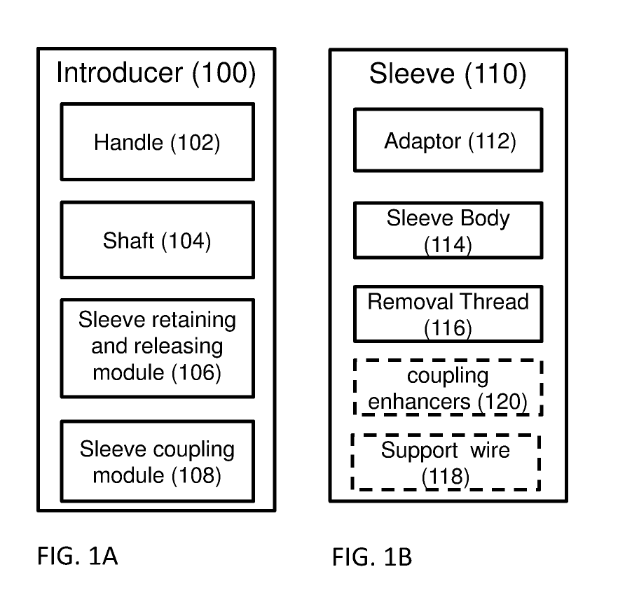

FIG. 1A is a schematic block diagram of an exemplary sleeve introducing device 100 according to an optional embodiment of the present invention. Sleeve introducing device 100 provides for introducing an airway tube lining sleeve 110, schematically depicted in FIG. 1B, into an airway tube 50 (not shown) along its internal surface such that the sleeve's external surface 114e is non-chemically coupled with the internal surface of airway tube 50.

Introducer 100 preferably comprises a handle 102, shaft 104, Sleeve retaining (receiving) and releasing module 106, and, a sleeve coupling module 108. Introducer 100 provides for associating with sleeve 110 along its shaft and for introducing the sleeve into airway tube 50 through its proximal end 50p and toward its distal end 50d.

Preferably introducer 100 comprises a handle 102 for manipulating introducer 100. Shaft 104 extends distally from the handle and is provided for associating with and/or receiving a lining sleeve member 110.

Optionally shaft 104 may be provided in the form of a hollow tube provided to maintain an open lumen along the length of airway tube 50. Optionally shaft 104 may comprise a recess 104r along its length for receiving sleeve 110. Optionally sleeve 110 may be associated within the shaft recess 104r to assume a minimal sleeve profile.

Preferably introducer 100 comprises a sleeve retaining and releasing module 106 that provides for securely retaining sleeve 110 during the introduction process and allows for releasing the sleeve 110 to allow for non-chemically coupling sleeve 110 onto the internal surface of airway tube 50. Optionally and preferably module 106 is disposed at the distal end of shaft 104. Optionally module 106 is controlled with at least one or more controllers and/or buttons 106e on handle 102, for example to retain or release and/or deploy sleeve 110 within airway tube 50.

Optionally sleeve 110 may be retained by module 106 in a plurality of optional manners for example including but not limited to mechanical clamping, suction, wrapping, the like, or any combination thereof.

Optionally once sleeve 110 is in place, preferably adjacent to the distal end of airway tube 50, sleeve 110 may be released and/or deployed with module 106 to releasing sleeve 110 from shaft 104. Optionally sleeve to 110 is deployed into the lumen of airway tube 50 in a gradual and directional manner preferably from the distal end of airway tube 50 to proximal end of airway tube 50.

Optionally if sleeve 110 is deployed within an airway tube 50 having a Murphy Eye at its distal end, for example as in an endotracheal tube, optionally sleeve 110 may be deployed proximally adjacent to the Murphy Eye so as to not cover the Murphy Eye with sleeve 110. Optionally sleeve 110 may be configured to include and/or account for the Murphy Eye.

Once sleeve 110 is deployed into the airway tube 50 lumen preferably sleeve coupling module 108 provides for urging the external surface of sleeve 110 onto the internal surface of airway tube 50. Optionally coupling module 108 is provided by mechanically smoothing and/or pressing the external surface of sleeve 110 onto the internal surface of airway tube 50. Optionally coupling module 108 may be provided in the form of a ring like structure that expands and urges sleeves 110 external surface onto airway tube 50, therein providing for non-chemically coupling the two surfaces.

Optionally coupling module 108 may comprise at least one or more arms that may be controllably extended from within the lumen of sleeve 110 to provide for urging sleeve 110 onto airway tube 50.

Optionally coupling module 108 may be configured to provide for unfolding sleeve 110 from a minimal profile configuration and to an expanded configuration and thereafter urging coupling of the expanded sleeve 110 with airway tube 50 along its internal surface.

Optionally coupling module 108 by urging and/or pressing the external surface of sleeve 110 onto the internal surface of airway tube 50 provides for the creation of non-chemically coupling the surfaces, optionally encouraging electrostatic forces and/or van der Waals forces to securely couple the two surfaces. Optionally the external surface of sleeve 110 may be configured so as to increase the coupling forces with airway tube 50. Optionally the materials forming sleeve 110 may be controlled so as to to increase the non-chemical coupling of sleeve 110 with airway tube 50.

FIG. 1B shows a schematic block diagram of sleeve 110 according to optional embodiments the sleeve comprising an adaptor 112, sleeve body 114, and removal thread 116. Optionally sleeve 110 may further comprise supporting thread and or wire 118. Optionally sleeve 110 may further comprise surface coupling enhancers 120.

Sleeve 110 is preferably provided in a tubular form that corresponds in its dimensions, particularly diameter and length, to the size of the airway tube 50 it is intended to be coupled with. Preferably at its proximal end 110p tube 110 is fit with a sleeve-airway tube adaptor 112 provided for securely associating sleeve 110 with the proximal and/or machine end of airway tube 50. Optionally adaptor 112 further facilitates coupling both to an airway tube 50 and a ventilation device.

Optionally adaptor 112 may be provided from a single unit. Optionally adaptor 112 may be provided from at least two subunits that may be associated or disassociated with one another.

Sleeve body 114 is preferably provided in tubular form having a distal end 114d and a proximal end 114p. Preferably proximal end 114d is securely fixed with adaptor 112. Distal end 114d is preferably provided to associate with the distal end of airway tube 50 about its distal end.

Optionally distal end 114d may be configured to mirror the structures provided by the distal end of airway tube 50. For example an airway tube 50 in the form of an Endotracheal tube (ETT) comprising a beveled end and a Murphy eye at its distal end may optionally utilize a sleeve 110 having a sleeve body 114 configured to include a beveled end and a Murphy Eye to correspond to the distal end of the ETT.

Optionally sleeve body 114 may be provided from optional biocompatible materials for example including but not limited to latex, silicone, medical grade polymers, biocompatible elastomers, medical grade silicone, Polyvinyl Chloride (PVC), PVC elastomers, fluorinated polymers (PTFE), polyolefin, polyurethane, Teflon, nylon, Nitrile butadiene rubber (NBR), soft PVC, polypropylene, the like, any mixture thereof, any hybrid thereof, any composition thereof, any combination thereof.

Optionally sleeve body 114 may be provided from biocompatible materials that allow it to have a wall thickness of about 0.2 mm. Optionally wall thickness may configured to be about 0.05 mm, 0.075 mm, 0.1 mm, 0.125, 0.15 mm, 0.0175 mm, 0.2 mm, 0.225 mm, 0.25 mm, 0.275 mm, 0.3 mm, 0.325 mm. 0.35 mm, 0.375 mm, 0.4 mm, 0.425 mm, 0.45 mm, 0.475 mm, 0.5 mm, 0.525 mm, 0.55 mm, 0.575 mm, 0.6 mm, 0.65 mm, 0.7 mm, 0.75 mm, 0.8 mm, 0.85 mm, 0.9 mm, 0.95 mm, 1.0 mm, or the like.

Sleeve body 114 comprises a wall 114w defining an inner surface 114i having an inner diameter and an external surface 114e having an outer diameter.

Preferably wall 114w comprises at least one sleeve removal thread, disposed along the tubes length. Removal thread 116 preferably provides for safely and seamlessly removing sleeve 110 from airway tube 50 after it is no longer useful and/or becomes contaminated with bio-burden. For example, sleeve 110 may be removed from airway tube 50 after its inner surface 114i has become soiled with bio-burden requiring its removal.

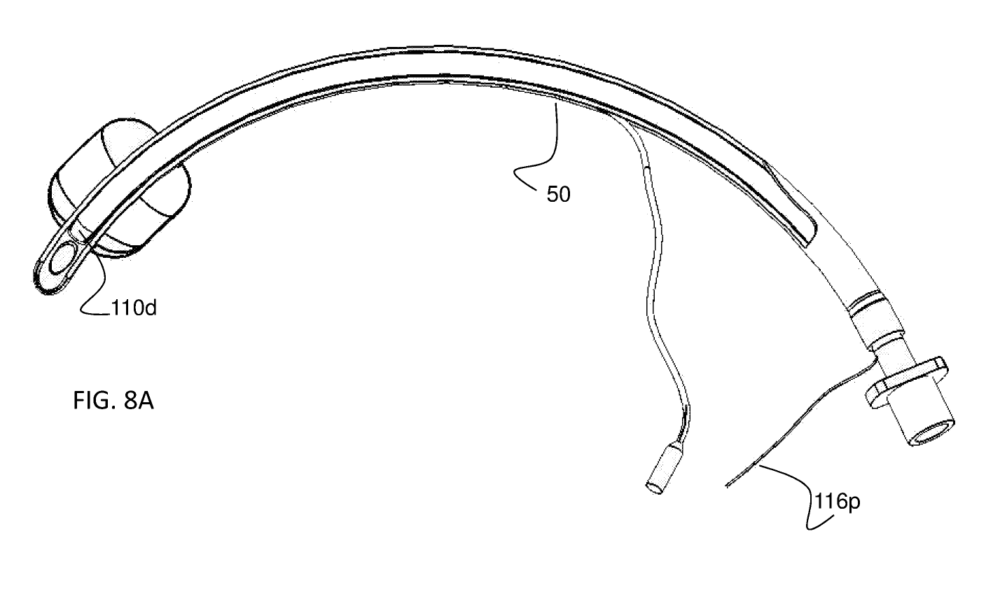

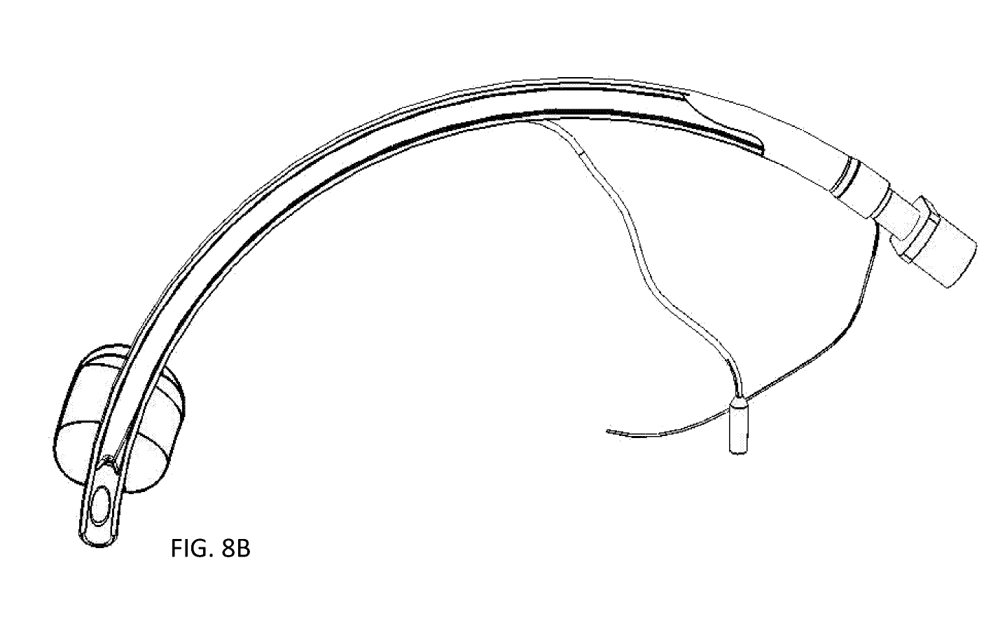

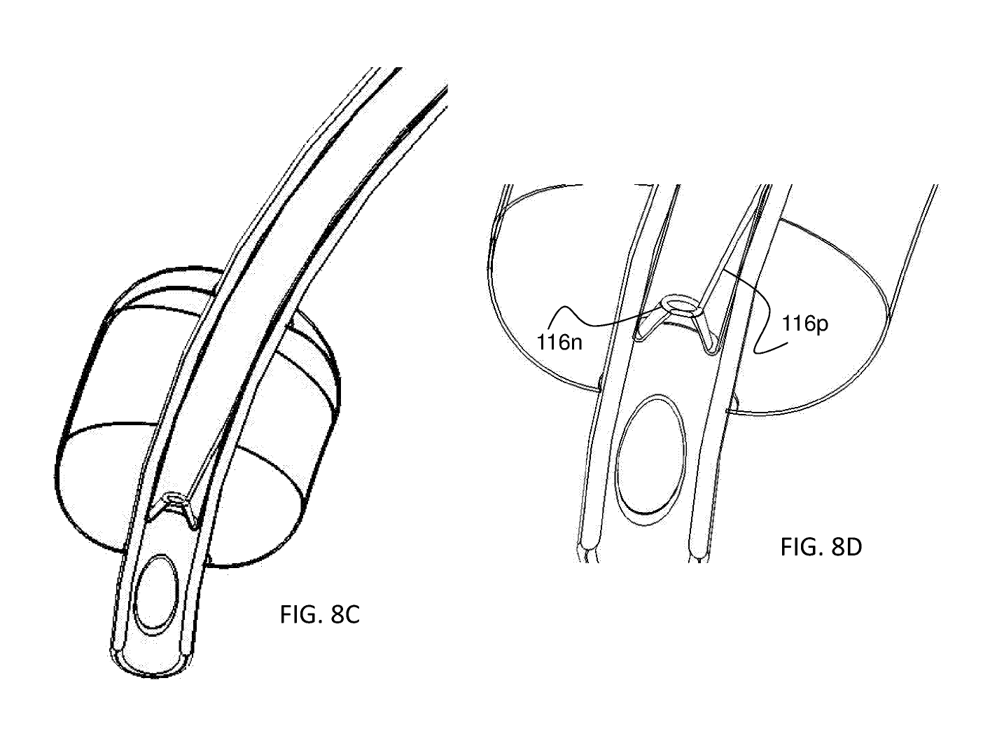

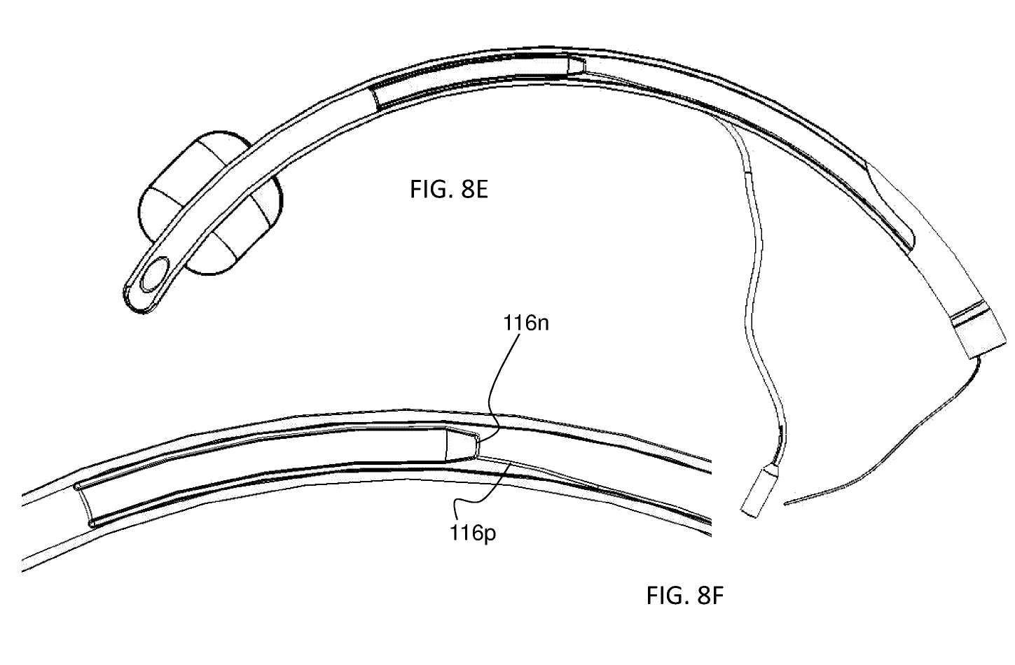

Optionally and preferably removal thread 116 is embedded within wall 114w from distal end 114d and exits through the proximal end 114p. Preferably removal thread 116 provides for safely removing and folding sleeve 110 in an inverted manner so as to prevent the fall off of bio burden particles and debris into the airway system while maintaining the patency of airway tube 50, as will be described with respect to FIG. 8A-F.

Optionally and preferably removal thread 116 may be associated with at least a portion of adaptor 112. Optionally removal thread 116 may be associated with a sub-unit of adaptor 112 such that the subunit may be pulled along with removal thread 116.

Optionally and preferably removal thread 116 is formed from a noose portion 116n and a pulling portion 116p, that provide a single removal thread 116 that is imbedded within the wall 114w of sleeve 110. Preferably noose to portion 116n is disposed at the distal end of removal thread 116.

Preferably the distal end 110d of sleeve 110 is fit with noose portion 116n within wall 114w. Preferably noose portion 116n provides for controllably reducing the diameter of sleeve 110 without fully closing the distal end of sleeve 110 therein maintaining an open lumen for sleeve 110 and airway tube 50. Optionally the diameter of sleeve 110 may be reduced by up to about 75% of the diameter of airway tube 50. Optionally the diameter of sleeve 110 may be reduced by up to about 50% of the diameter of airway tube 50. Optionally the diameter of sleeve 110 may be reduced by up to about 25% of the diameter of airway tube 50. Optionally the diameter of sleeve 110 may be reduced by up to about 15% of the diameter of airway tube 50.

Optionally noose portion 116n may be provided with at least one or more stopping members along its perimeter to provide for controlling the noose closure.

Optionally noose portion 116n may be controlled by pulling the pulling portion 116p of removal thread 116 proximally to reduce the diameter of sleeve 110 in a controlled manner.

Most preferably removal thread 116 provides for the disassociation and removal of sleeve 110 from airway tube 50 while maintain the patency of sleeve 110 and airway tube 50 allowing for continued ventilation. Preferably removal thread 116 provides for the disassociation and removal of sleeve 110 from airway tube 50 while sleeve 110 is maintained intact with reduced risk of bio-burden leakage into the lower airway tract.

Optionally sleeve 110 may be fit with optional support frames 118 for example provided from optional biocompatible materials for example including but not limited to support wires, filaments, fibers, threads or the like, or any combination thereof. Optionally support frame 118 may be disposed within sleeve walls 114w and may be provided in optional configuration and shapes for example including but not limited to helical, double helix, circular, mesh, grid-like or the like. Optionally support frame 118 may be provided about only a portion of sleeve 110. Optionally support frame 118 may be provided along the length of sleeve 110. Optionally support frame 118 may be provided along the length of sleeve 110 at select portions of sleeve 110, for example at the distal end, along the portion corresponding to the airway tube curvature.

Optionally sleeve 110 may be fit with sleeve coupling enhancers 120 disposed along the external surface of sleeve 110. Most preferably enhancers 120 are not chemical enhancers and do not include a fluid. Optionally and preferably enhances 120 provide for increasing the surface area of external surface 114e. Optionally enhances 120 may be shaped to increase the holding forces between the internal surface of airway tube 50 and the external surface 114e of sleeve 110. Optionally enhancers 120 may be provided form a plurality of shapes for example including but not limited to concave, convex, rod-like extensions, the like or any combination thereof.

Preferably sleeve coupling enhancers 120 provide for increasing the holding and/or coupling forces acting between the internal surface of airway tube 50 and the external surface 114e of sleeve 110. Optionally the external surface 114e may be configured to included coupling enhances 120 that provide to increase the van der Waals forces and/or the electrostatic forces acting between the internal surface of airway tube 50 and the external surface 114e of sleeve 110.

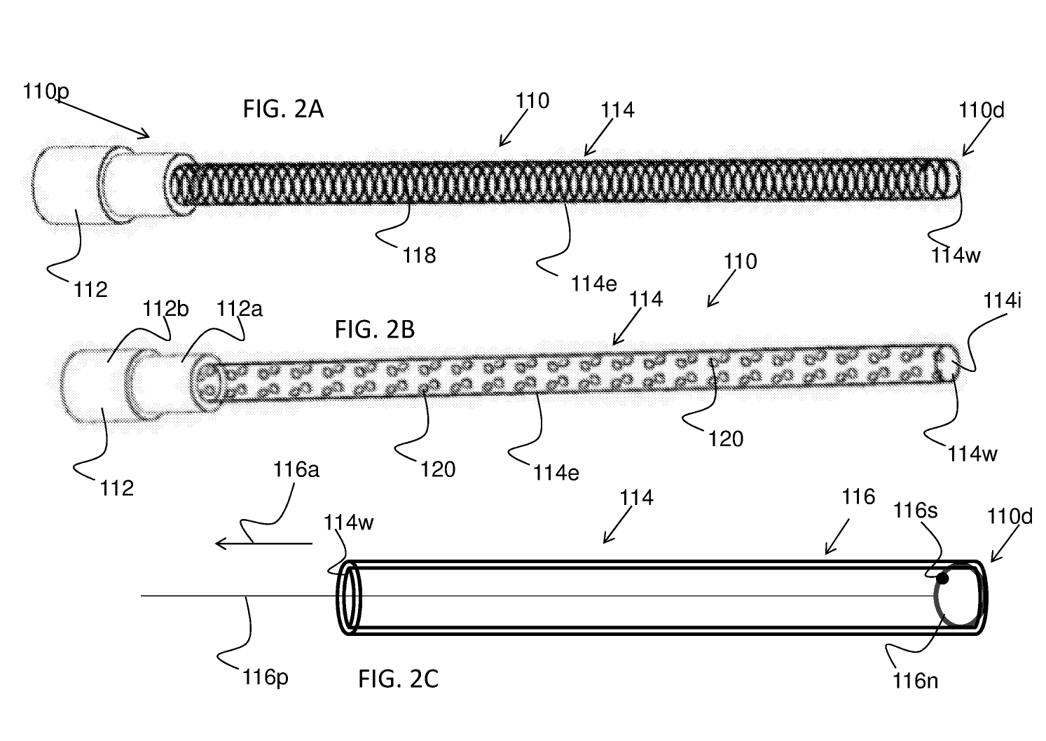

FIG. 2A shows a schematic illustration of an optional airway tube lining sleeve 110, comprising an adaptor 112 and a sleeve body 114 according to an optional embodiment of the present invention. FIG. 2A also shows an optional support frame 118 disposed within the wall 114w of sleeve body along the length of sleeve 110.

FIG. 2B shows a schematic illustration of an optional airway tube lining sleeve 110, comprising an adaptor 112 and a sleeve body 114 and further comprising coupling enhancers 120 as previously described that are disposed along external surface 114e according to an optional embodiment of the present invention. FIG. 2B shows optional enhancers 120 provided in the form of concave members disposed along the length of sleeve 110 about 114e. FIG. 2B further shows adapter 112 that is provided from two subuints 112a, 112b that may be associated and disassociated from one another, optionally to pull removal thread (not shown) 116 proximally.

FIG. 2C shows a schematic illustration of a lining sleeve 114 showing the removal thread 116 disposed within wall 114w. Removal thread comprising a noose portion 116n disposed at the distal end of sleeve body 114 and a pulling portion 116p disposed along its length. Preferably when removal thread 116 is pulled proximally in the direction of arrow 116a along pulling portion 116p, noose portion 116n closes to a controllable degree, as previously described. Optionally the degree to which noose portion closes and therein reducing the sleeve diameter at its distal end 110d, may optionally be determined by the location of a stopping member 116s. Preferably as removal thread 116 is pulled in direction 116a sleeve body 114 disassociates from the internal surface of airway tube 50 in an inverted manner as will be shown in FIG. 8A-F so as to both reduce the risk of bio-burden leakage and maintain the patency of sleeve 110 and therein airway tube 50.

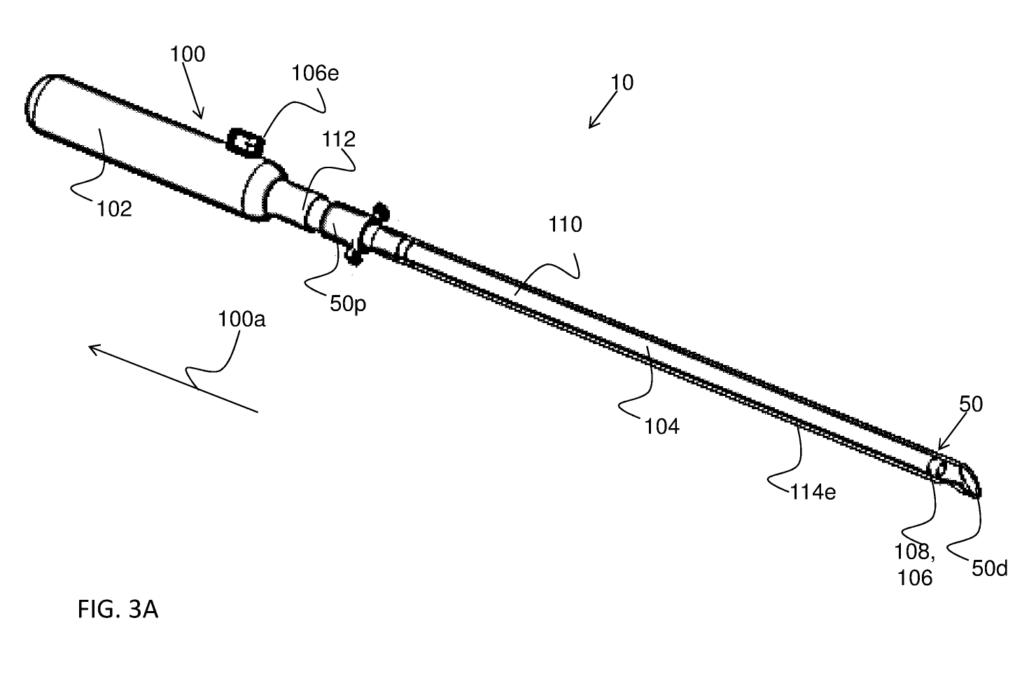

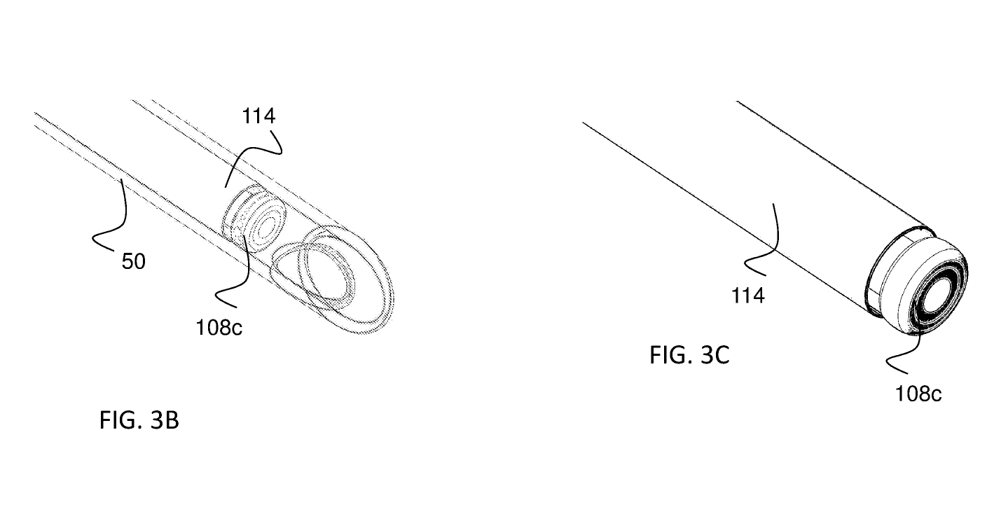

FIG. 3A shows a schematic illustration of an airway tube inner sleeve introducing system 10 including the sleeve 110 disposed about the shaft of an introducer 100 that is being introduced into airway tube 50 shown in the form of an endotracheal tube.

Sleeve introducing device 100 comprising manipulating handle 102 is inserted into airway tube 50 from its proximal end (machine end) to its distal end near the Murphy Eye. Sleeve 110 is associated along shaft 104 that is retained with sleeve retaining module 106 having a controller 106e disposed on handle 102. Preferably once sleeve 110 reaches the distal end 50d of airway tube 50, sleeve 110 is released from shaft 104 optionally and preferably with the assistance of retaining and releasing module 106 which thereafter deploys coupling module 108, schematically shown in a ring like structure. Coupling module 108 is preferably provided to urge external surface 114e of sleeve body 114 along the internal surface of airway tube 50. Optionally module 108 shown in the form of a ring structure is expanded to snuggly fit against the internal surface of sleeve body 114 to urge external surface 114e of sleeve body 114 against the internal surface of airway tube 50. Optionally module 108 may be controllably expanded to provide and apply the appropriate pressure between sleeve body 114 and internal surface of airway tube 50.

Optionally and preferably module 108 provides for coupling of sleeve 110 to airway tube 50 when extracting introducer 100 proximally, as shown with arrow 100a, from the lumen of airway tube 50, wherein as device 100 is extracted the coupling member 108c in the form of a ring structure urges the external surface of sleeve body 114 against the internal surface of airway tube 50, as shown in more detail in FIG. 3B-C.

FIG. 3B-C show a close up view of optional module 108 depicted in FIG. 3A. Optionally module 108 may be controlled with a controlling button (not shown) disposed on handle 102. Optionally controlling module 108 may be facilitated with the controller 106e of module 106.

Optionally control module 108 may comprise a moveable member, for example a ring-ling member that may be moved along shaft 104 between its distal end and proximal end to couple to facilitate coupling of sleeve 110 to airway tube 50.

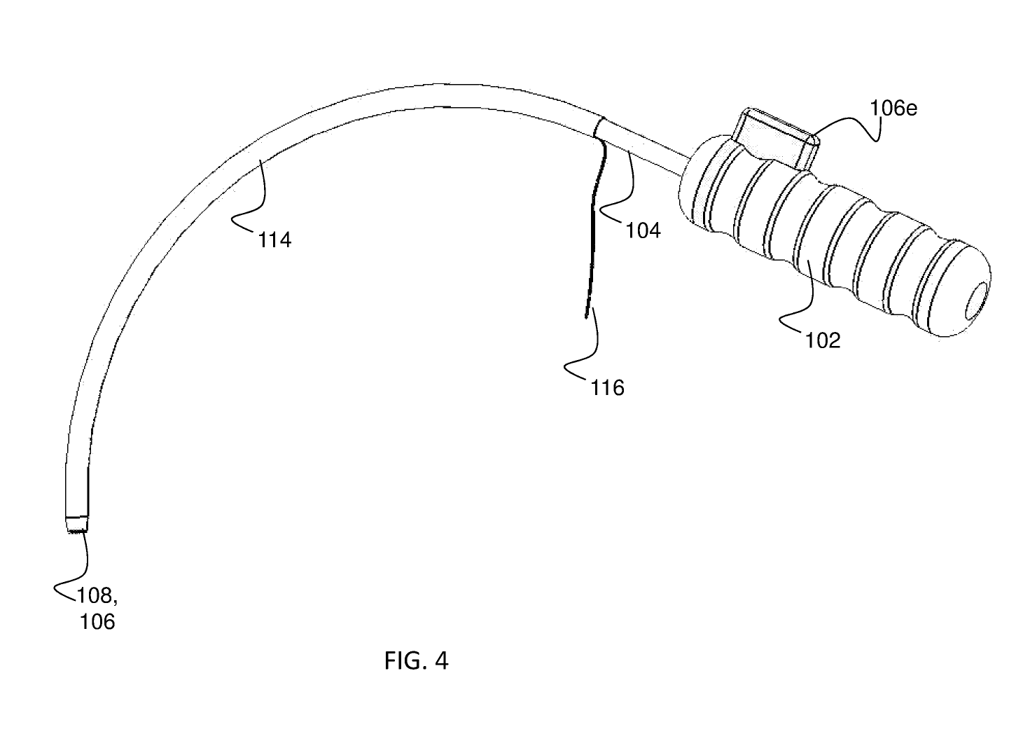

FIG. 4 shows a further depiction of introducing system 10 where as shown in FIG. 3 however with airway tube 50 removed. FIG. 4 shows an optional introducing device 100 comprising handle 102, shaft 104 that is associated with sleeve body 114, a retaining and releasing module 106 and coupling module 108 as previously described.

FIG. 4 shows sleeve body 114 with removal thread 116 exposed as adaptor 112 is not shown.

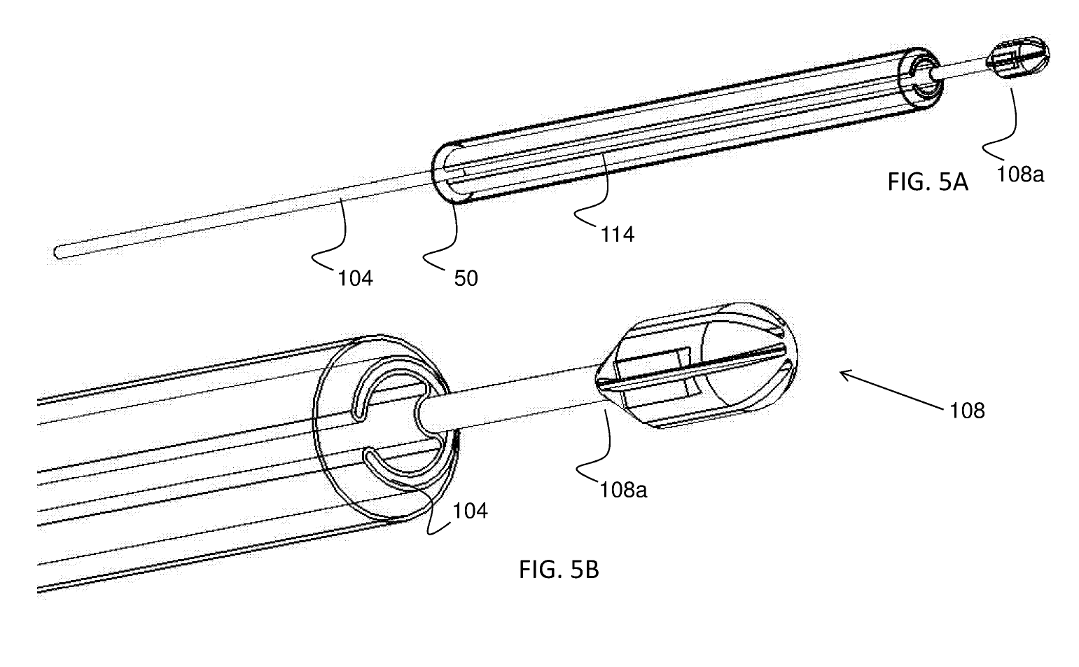

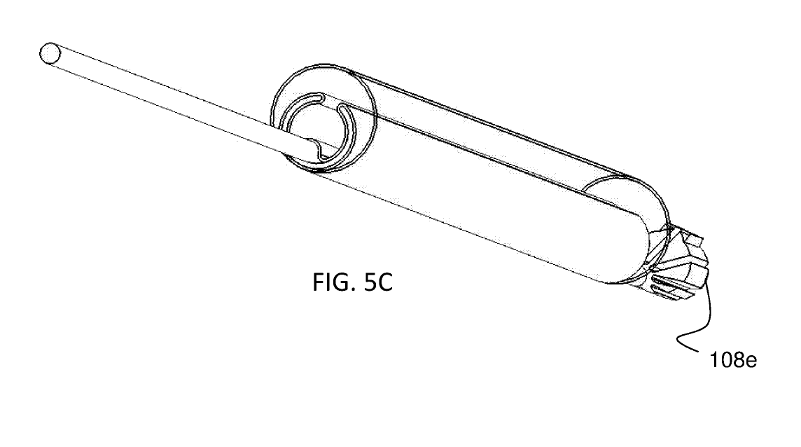

FIG. 5-7 show further depiction of optional sleeve retaining and releasing modules 106 and coupling modules 108 provided to urge sleeve 110 onto airway tube 50 along its internal surface therein lining airway tube 50.

FIG. 5A-C show an optional coupling module 108, for a sleeve body 114 that is disposed about an introducer shaft 104 in a low profile configuration within airway tube 50 prior to deployment and coupling with module 108. Sleeve 114 is wrapped about shaft 104 assuming a “C” shaped low profile configuration as shown, FIG. 5B.

Optionally sleeve low profile configuration is provided with module 106 to retain sleeve body 114 onto shaft 104 in the low profile “C” configuration as shown prior. Optionally module 106 provides for retaining sleeve 114 in a low profile configuration utilizing mechanical means for pressing sleeve 114 onto shaft 104. Optionally module 106 may utilize suction means for retaining sleeve 114 onto shaft 104.

Optionally shaft 104 may be configured to provide for retaining sleeve 114 in a low profile configuration utilizing mechanical means for pressing sleeve 114 onto shaft 104. Optionally shaft 104 may be configured to include a recess along its length to facilitate receiving and retailing sleeve body 114 in a low profile configuration.

Coupling module 108 shown comprises a low profile configuration 108a utilized when sleeve 110 is introduced with device 100 into airway tube 50. Once sleeve body 114 is placed at its most distal location within airway tube 50, sleeve body is released from shaft 104 via retaining and releasing module 106 so as to allow coupling module 108 to urge sleeve body 114 onto the internal surface of airway tube 50.

Coupling module 108 may comprises a low profile coupling member 108a that is expanded to the expanded profile 108e to allow for coupling sleeve 110 onto the internal surface of airway tube 50. Optionally the release of sleeve body 114 from shaft 104 may trigger or be performed simultaneously with the expanding of coupling module 108 to assume the expanded form 108e, shown in FIG. 5C.

Optionally the expanded form 108e may optionally provide for expanding sleeve 114 from a low profile configuration to its expanded configuration as it urges sleeve body 114 onto airway tube 50 while device 100 is moved proximally.

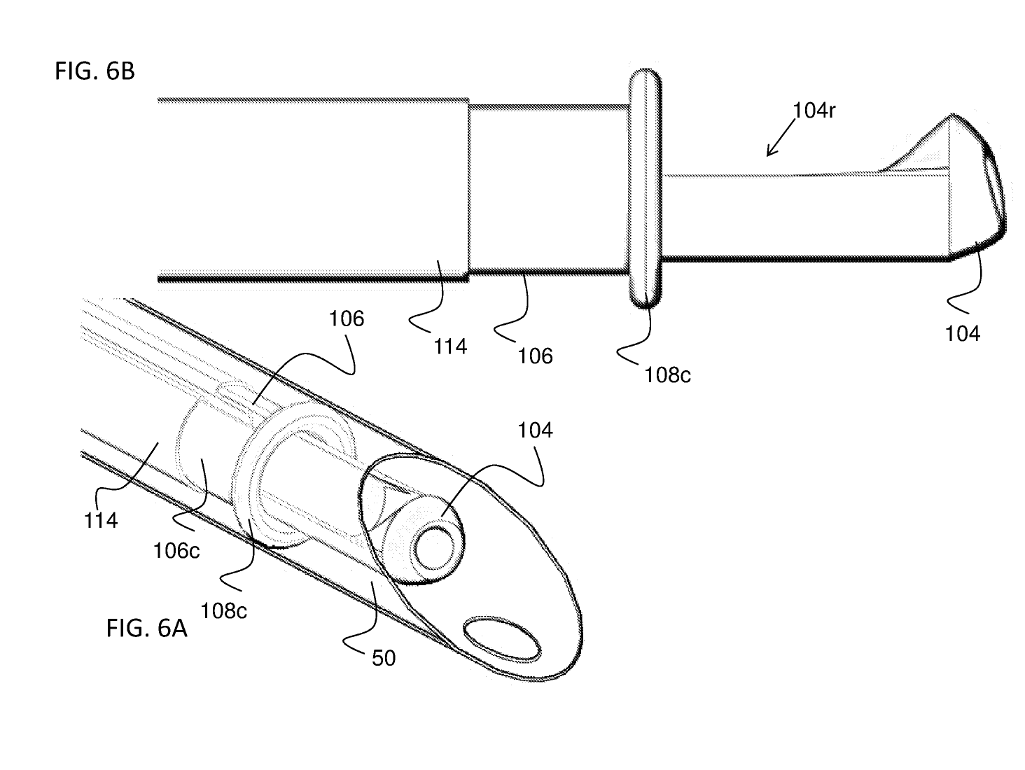

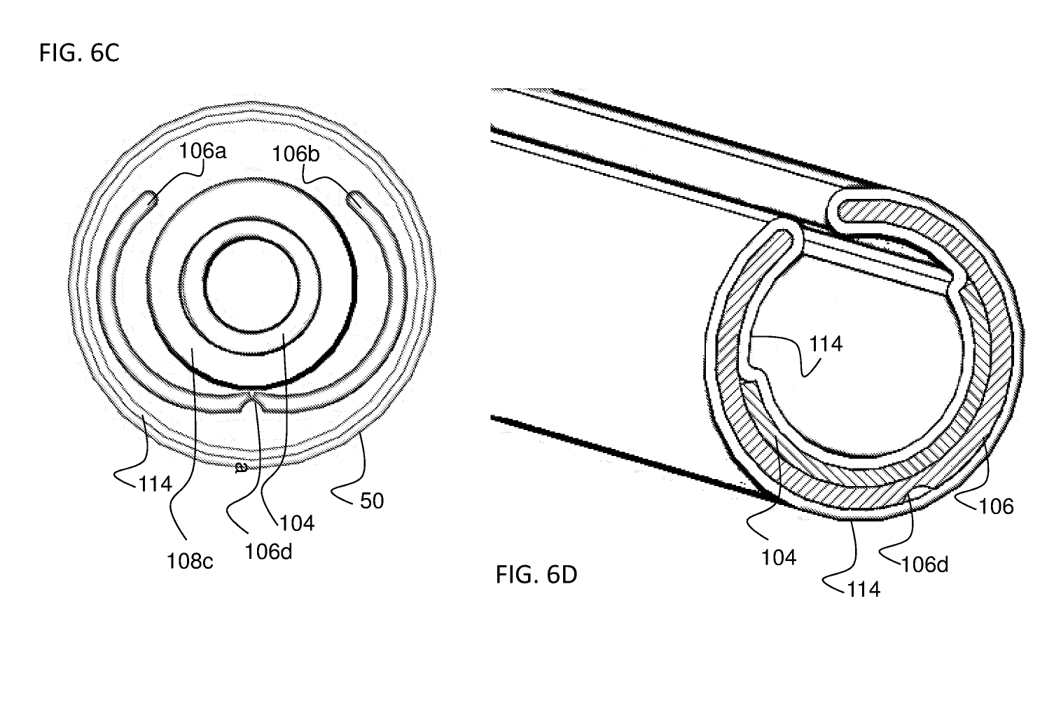

FIG. 6A-D shows a further optional configuration of device 100 showing coupling of sleeve body 114 onto the internal surface of airway tube 50.

FIG. 6A shows the distal end of an optional introducing device 100 having a shaft 104 that comprises a hollow recess provided for housing and associating with retaining and releasing module 106, coupling module 108 and sleeve body 114. Shaft 104 is configured to have a central recess for concentrically receiving and layering modules 106, 108 and sleeve body 114. Preferably sleeve body 114 is disposed in a folded and/or minimal profile configuration about the shaft defining receiving module 106, for example as shown in FIG. 6A, 6D. Optionally sleeve body 114 may be configured along retaining module 106 to assume a low profile “C” shaped configuration for example as shown in FIG. 6D.

Optionally sleeve body 114 is retained with module 106 via a central shaft having a recess along its length for receiving and retaining sleeve body 114 in a minimal profile. Optionally and preferably retaining and releasing module 106 is provided in the form of an elongated shaft “C” shaped shaft 106c having an opening for receiving sleeve body 114.

Optionally shaft 106c may be formed from at least two flanking arms 106a, b that are coupled with a pivot 106d, as shown in FIG. 6C. Pivot 16d provides for pivoting arms 106a and 106b allowing them to move away from one another when releasing sleeve body 114 from its “C” shaped minimal profile and/or closed configuration allowing sleeve body 114 to assume the open configuration. Optionally and preferably arms 106a, b further provide for pressing sleeve body 114 against internal surface of airway tube 50.

Optionally and preferably shaft 106c is coupled with an optional coupling member 108c that is disposed at the distal end of shaft 106c. Preferably coupling member 108c provided in the form of a ring like structure allows for smoothing and urging the open configuration of sleeve body 114 onto the internal surface of airway tube 50, therein contributing to their coupling.

FIG. 6D shows a cross-sectional view of introducer 100 fit with sleeve body 114 in the folded “C” state shape, showing the layered configuration provided by the employment of a shaft 104 having a recess 104r, that is associated with a C shaped shaft 106c forming a sleeve retaining and releasing module that allows to both to receive and retain the folded configuration of sleeve body 114 while allowing for unfolding sleeve body 114 to its open configuration with the arms 106a,b forming shaft 106c.

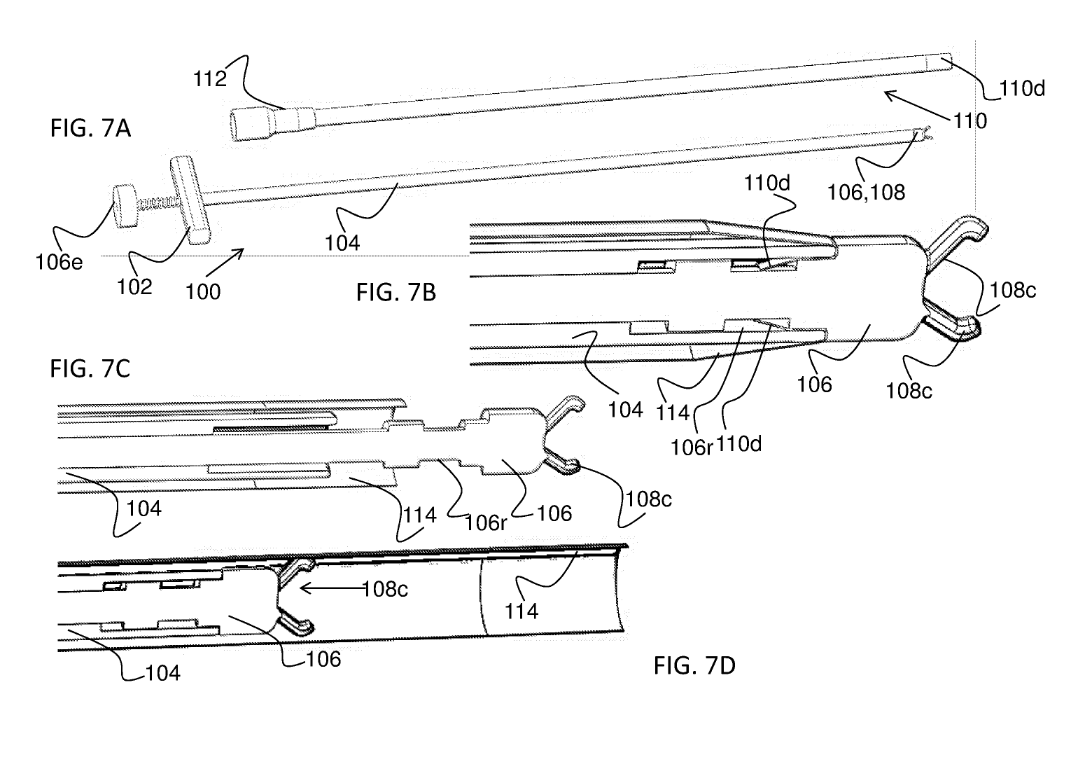

FIG. 7A-D shows a further optional configuration of introducing device 100 showing the coupling of sleeve body 114 onto the internal surface of airway tube 50.

FIG. 7A shows sleeve 110 prior to loading onto introducing device 100. Device 100 comprises a handle 102, shaft 104, a sleeve retaining and releasing module and a sleeve coupling module. Handle 102 is provided in a simplified form having a sleeve releasing controller 106e. Sleeve releasing controller 106e provides for controlling the retaining and releasing sleeve 110 functions of module 106. Optionally modules 106 and 108 are disposed at the distal end of shaft 104.

Sleeve 110 may be loaded onto shaft 104 through adapter 112 where distal end 110d of sleeve 110 is associated with module 106, shown in greater detail in FIG. 7B. Preferably sleeve distal end 110d is folded onto a recess in module 106 so as to allow the device 100 to retain and hold sleeve 110 in a fixed position while it is being introduced into airway tube 50. As shown in FIG. 7B the distal end 110d is folded over into retaining recess 106r of module 106 and is maintained in position against shaft 104. Accordingly to FIG. 7B shows the sleeve 110 and introducer 100 configuration when sleeve 110 is introduced into the lumen of an airway tube 50 prior to releasing and coupling lining sleeve 110 with the internal surface of airway tube 50.

FIG. 7C depicts the release of lining sleeve 110 from module 106 and prior to coupling with coupling module 108. Sleeve 110 is released from module 106 by utilizing controller 106e to push module 106 assembly distally therein releasing sleeve 110. Thereafter coupling module 108 is utilized to urge sleeve 110 onto an optional airway tube 50. During coupling, as depicted in FIG. 7D coupling member 108c showing a non limiting configuration in the form of two arms, optionally a ring like structure may be similarly employed, urge coupling of sleeve body 114 onto airway tube 50 (not shown) as shaft 104 is drawn proximally out of airway tube 50 leaving that is coupled with sleeve 110.

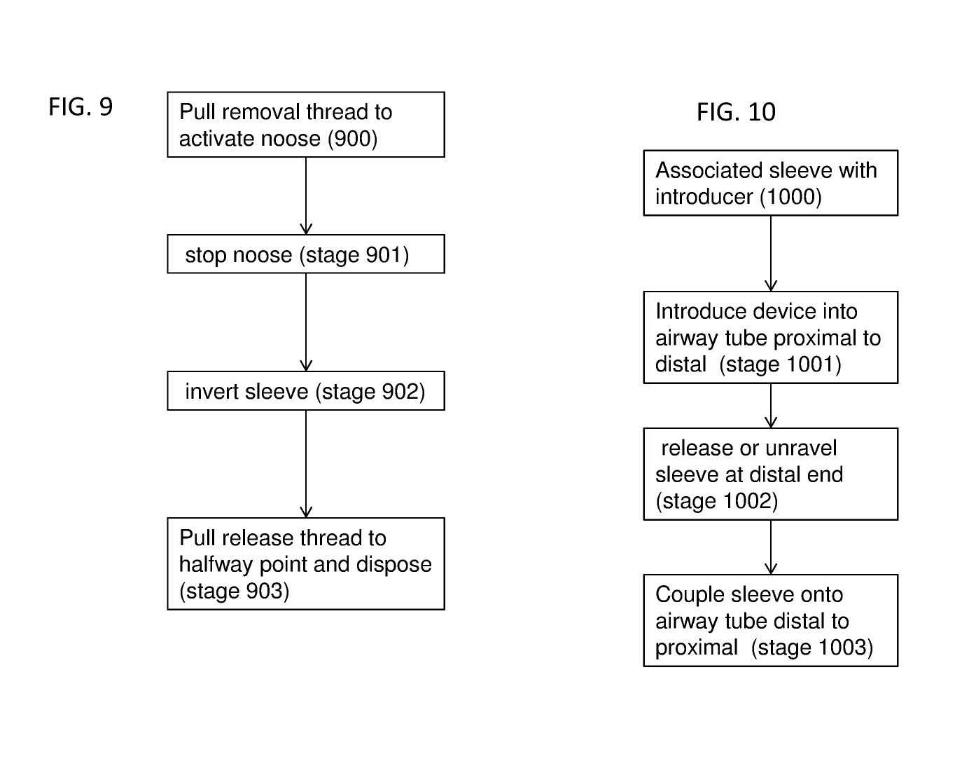

FIG. 8A-F show schematic illustrations of the various stages of removing an airway tube lining sleeve 110 from an airway tube 50, shown in the optional form of an endotracheal tube. Most preferably during removal and/or extraction of sleeve 110 the patency of airway tube is maintained. The removal stages are also shown in flowchart of FIG. 9

FIG. 8A shows an initial removal stage as depicted in stage 900 of FIG. 9 where removal thread 116 is pull along pull portion 116p activating noose portion 116n to controllably reduce the sleeve's diameter at the sleeve 110 distal portion 110d.

Next in stage 901 and shown in FIG. 8B, noose portion 116n is stopped to maintain an open lumen. Optionally and preferably noose portion 116n comprises an internal stopping member (not shown) to ensure that patency of sleeve 110 is maintained.

Next in stage 902 the sleeve inversion process is initiated by further pulling release thread 116 on its pulling portion 116p. A close up view of the noose portion 116n and an initial view of the inversion of sleeve 110 is provided in FIG. 8C-D

Next in stage 903 as shown in FIG. 8E-F sleeve 110 is inverted as it is maneuvered proximally. Optionally and most preferably when sleeve 110 reaches a halfway point along the airway tube 50 or for example where noose portion 106n approaches adaptor 112. At this point the used sleeve 110 may be removed from the airway tube.

FIG. 10 shows a schematic flow chart summarizing the method for introducing a sleeve 110 with an introducing device 100 for ling the internal surface of an optional airway tube, for example as described and shown in FIG. 1-7.

First in stage 1000 sleeve 110 and introducing device 100 are associated with one another optionally and preferably utilizing retaining and releasing module 106, for example as previously described with respect to FIG. 7A-D.

Next in stage 1001 the sleeve loaded introducing device 100 is introduced into the internal lumen of airway tube 50.

Next in stage 1002 once sleeve 110 and device 100 reach the distal end 110d, sleeve 110 is released and/or unraveled with module 106, as previously described.

Next in stage 1003 the external surface of released sleeve 110 is urged onto the internal surface of airway tube 50 with optional configuration of coupling module 108.

While the invention has been described with respect to a limited number of embodiment, it is to be realized that the optimum dimensional relationships for the parts of the invention, to include variations in size, materials, shape, form, function and manner of operation, assembly and use, are deemed readily apparent and obvious to one skilled in the art, and all to equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not described to limit the invention to the exact construction and operation shown and described and accordingly, all suitable modifications and equivalents may be resorted to, falling within the scope of the invention.

Having described a specific preferred embodiment of the invention with reference to the accompanying drawings, it will be appreciated that the present invention is not limited to that precise embodiment and that various changes and modifications can be effected therein by one of ordinary skill in the art without departing from the scope or spirit of the invention defined by the appended claims.

Further modifications of the invention will also occur to persons skilled in the art and all such are deemed to fall within the spirit and scope of the invention as defined by the appended claims.

While the invention has been described with respect to a limited number of embodiments, it will be appreciated that many variations, modifications and other applications of the invention may be made.

1) A device for introducing an airway tube sleeve for lining an airway tube the device comprising: a handle for manipulating the device into an airway tube, an introducing shaft, said shaft extending distally from said handle and provided for associating with said sleeve; a sleeve retaining and releasing module for disassociating said sleeve from said shaft; and a sleeve coupling module disposed about the distal end of said shaft provided for facilitating coupling said sleeve onto the inner wall of said airway tube.

2) The device of claim 1 wherein said sleeve coupling module comprises a coupling member provided for urging the external surface of said airway tube lining sleeve onto the inner wall of said airway tube therein non-chemically coupling the surfaces.

3) The device of claim 1 wherein said airway tube and airway tube lining sleeve are coupled by way of electrostatic forces and/or van der Waals forces.

4) (canceled)

5) The device of claim 1 wherein said coupling member is provided in the form of a ring that may assume a small diameter when introduced and an expanded diameter when coupling said airway tube lining sleeve with said airway tube

6) The device of claim 5 wherein said ring member is provided from at least two semi-circular members.

7) The device of claim 1 wherein said coupling member is provided in the form of a claw having at least two manipulating fingers.

8) The device of claim 1 wherein said coupling member assumes a small profile when introducing said airway tube lining sleeve and an expanded profile when coupling said airway tube lining sleeve.

9) The device of claim 1 wherein said coupling member comprises a plurality of radially extending projection.

10) The device of claim 1 wherein a proximal portion of said sleeve retaining and releasing module provides for unfolding said airway tube lining sleeve.

11) The device of claim 10 wherein said proximal portion provides for releasing said airway tube lining sleeve from said introduce shaft.

12) The device of claim 1 wherein a distal portion of said coupling module provides for urging the external surface of said airway tube lining sleeve onto the inner wall of said airway tube

13) The device of claim 1 wherein said introducing shaft comprises a recess along its length provided for receiving and housing said airway tube lining sleeve in a minimal profile configuration.

14) The device of claim 1 wherein said sleeve retaining and releasing module comprises a recess for retaining at least a portion of the distal portion of said airway tube lining sleeve.

15) An airway tube lining sleeve forming a substantially tubular body having an open lumen defined between a distal end and a proximal end, said proximal end affixed with an adaptor for coupling both with the machine end of an airway tube and with a ventilation device; said tubular body defining a wall, said wall having at least one intrinsic removal thread spanning the length of said tubular body and wherein said removal thread forms a noose spanning the perimeter of the sleeve's distal end; said noose provided for reducing the circumference of said distal end and allowing for the inversion of said lining sleeve during removal with said removal thread and wherein said wall defines an inner surface and an outer surface, wherein said outer surface is provided for coupling with the internal surface of an airway tube; and wherein said outer surface comprises coupling enhancers to enhance coupling between said lining sleeve and said airway tube.

16) (canceled)

17) The airway tube lining sleeve of claim 15 wherein said wall further comprises supportive wire disposed along the length of said tube body.

18) (canceled)

19) (canceled)

20) The airway tube lining sleeve of claim 15 wherein said coupling enhancers are provided in the form of protrusions extending from said external sleeve surface.

21) The airway tube lining sleeve of claim 20 wherein said protrusions assume a concave-external surface or convex external surface.

22) (canceled)

23) (canceled)

24) (canceled)

25) (canceled)

26) (canceled)

27) (canceled)

28) (canceled)

29) (canceled)

30) (canceled)

31) (canceled)

32) (canceled)

33) (canceled)

34) A system for introducing an airway tube lining sleeve into an airway tube wherein said airway tube is associated with a patient, the system comprising:
a) a sleeve introducing device for introducing an airway tube sleeve for lining an airway tube the device comprising: a handle for manipulating the device into an airway tube, an introducing shaft, said shaft extending distally from said handle and provided for associating with said sleeve; a sleeve retaining and releasing module for disassociating said sleeve from said shaft; and a sleeve coupling module disposed about the distal end of said shaft provided for facilitating coupling said sleeve onto the inner wall of said airway tube; and
b) and an airway tube lining sleeve forming a substantially tubular body having an open lumen defined between a distal end and a proximal end, said proximal end affixed with an adaptor for coupling both with the machine end of an airway tube and with a ventilation device; said tubular body defining a wall, said wall having at least one intrinsic removal thread spanning the length of said tubular body and wherein said removal thread forms a noose spanning the perimeter of the sleeve's distal end; said noose provided for reducing the circumference of said distal end and allowing for the inversion of said lining sleeve during removal with said removal thread and wherein said wall defines an inner surface and an outer surface, wherein said outer surface is provided for coupling with the internal surface of an airway tube; and wherein said outer surface comprises coupling enhancers to enhance coupling between said lining sleeve and said airway tube.

35) A method for introducing an airway tube lining sleeve into an airway tube associated with a patient the method comprising:
a) Associating an airway tube lining sleeve with an introducing device according claim 1;
b) Introducing said shaft into the proximal end of said airway tube through to the distal end of the airway tube;
c) Releasing said airway tube lining sleeve from said introducing device utilizing said releasing module; and
d) Non-chemically coupling said sleeve's external surface with said airway tube internal surface utilizing said sleeve coupling module to urge said external surface onto said airway tube internal surface from said distal end to said proximal end, with said extracting module and coupling module, as said introducing device is extracted proximally out of said airway tube.

36) (canceled)

37) The device of claim 1 wherein said airway tube is selected from the group consisting of: Endotracheal tube (ETT), endobronchial tube, tracheostomy tubes, laryngeal tubes, nasopharyngeal tubes, single lumen tubes, double lumen tubes, multi-lumen tubes.

38) (canceled)

39) (canceled)
